(19) United States
(12) Patent Application Publication
(10) Pub. No.: US 2003/0045852 A1
(43) Pub. Date: Mar. 6, 2003

(54) Disposable hygiene protection pad

(76) Inventor: Tanya L. Esselburn, Evansville, IN (US)

Correspondence Address: BOWERS HARRISON LLP, GARY K. PRICE, ESP., 25 RIVERSIDE DRIVE, PO BOX 1287, EVANSVILLE, IN, 47706-1287, US

(21) Appl. No.: 10/222,563

(22) Filed: Aug. 15, 2002

Related U.S. Application Data
(60) Provisional application No. 60/285,843, filed on Aug. 29, 2001.

(51) Int. Cl. A61F 13/15; A61F 13/20

(52) U.S. Cl. 604/385.14; 604/387

(57) ABSTRACT

A unique disposable hygiene protection product, namely a feminine hygiene protection pad that includes a longer design to accommodate the need that full-figured women have for a feminine hygiene napkin that covers more area than the current smaller designs. Further, the hygiene protection pad of the present invention includes a plurality of thin layers of absorbent hygiene pads, each of said pads are releasably interlocked with the other, so that the wearer may simply peel away the top layer pad and have remaining pad layers for protection underneath. This eliminates the inconvenience of carrying extra pads with the wearer. The present invention would allow the user to simply peel away a thin top layer of absorbent protection and still have other layers of fresh sanitary protection underneath.

### CROSS REFERENCES TO RELATED APPLICATIONS

U.S. Provisional Application for Patent 60/285,843, filed Aug. 29, 2001, with title, “Disposable Hygiene Protection Pad” which is hereby incorporated by reference. Applicant claims priority pursuant to 35 U.S.C. Par. 119(e)(i).

Statement as to rights to inventions made under Federally sponsored research and development: Not Applicable

### BACKGROUND OF THE INVENTION

1. Field of the Invention

This invention relates to a unique multi-layer disposable hygiene pad that includes a longer design which allows and accommodates the need that full-figured women have for a feminine hygiene napkin that covers more area than the current smaller designs. Said hygiene pad includes a plurality of thin layers of absorbent hygiene pads, each of said pads are releasably interlocked with the other, so that the wearer may simply peel away the top layer pad and have remaining pad layers for protection underneath. In addition, each of the layers of the multi-layer pad are interlocked around the outer edge of the layer, and adhesively connected at the ends.

2. Brief Description of Prior Art.

Feminine sanitary pads come in a wide variety of shapes and sizes. Such pads are externally worn by the user, and generally, have never successfully provided complete absorbency, or prevention from leakage. In addition, such pads require frequent changing which requires the wearer to carry extra pads. This is both inconvenient and costly for the wearer. For those reasons, known feminine hygiene pads have not successfully addressed the problem of providing complete and continuous hygienic control to a wearer.

This application in general relates to a disposable hygiene protection product, namely a feminine hygiene protection pad that has a longer design to cover more area for improved absorbency and to prevent leakage, and comprising a plurality of thin layers of absorbent hygiene pads, each of said pads are releasably interlocked with the other, so that the wearer may simply peel away the top layer pad and have remaining pad layers for protection underneath.

As will be seen from the subsequent description, the preferred embodiments of the present invention overcome shortcomings of prior art.

### SUMMARY OF THE INVENTION

The present invention in general relates to improvements in feminine hygiene pads that include a longer design to accommodate the need that full-figured women have for a feminine hygiene napkin that covers more area than the current smaller designs. Further, the hygiene protection pad of the present invention includes a plurality of thin layers of absorbent hygiene pads, each of said pads are releasably interlocked with the other, so that the wearer may simply peel away the top layer pad and have remaining pad layers for protection underneath. This eliminates the inconvenience of carrying extra pads with the wearer. The present invention would allow the user to simply peel away a thin top layer of absorbent protection and still have other layers of fresh sanitary protection underneath.

### BRIEF DESCRIPTION OF THE DRAWINGS

FIG. 1 is a perspective view of the present invention, a multi-layer disposable hygiene protection pad.

FIG. 2 illustrates a side view of the protection pad of FIG. 1, and specifically illustrates the interlocking means.

### DESCRIPTION OF THE PREFERRED EMBODIMENT

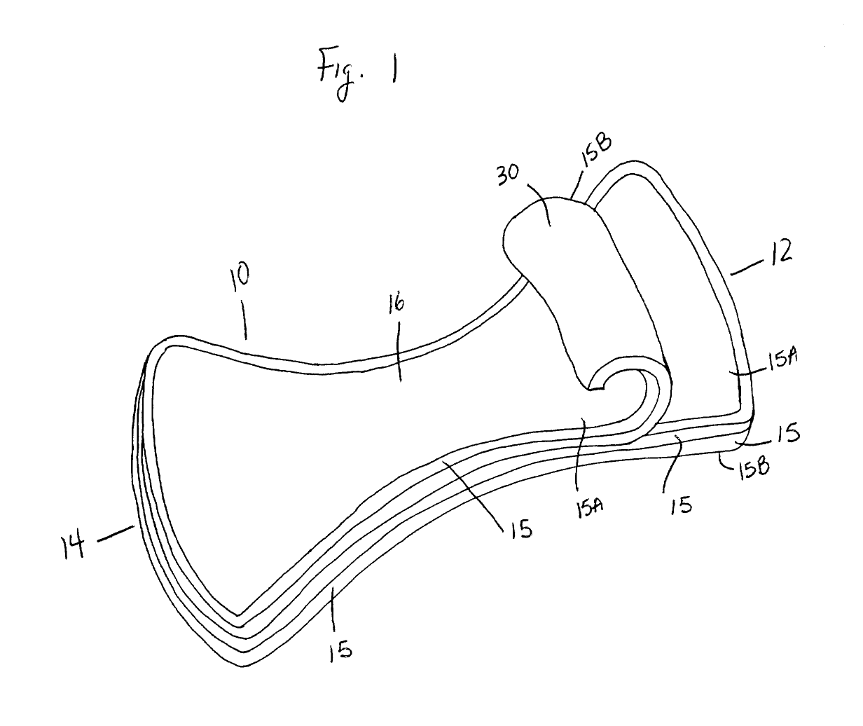

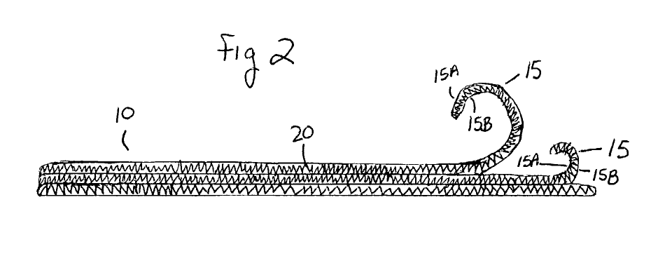

FIGS. 1 and 2 illustrate a preferred embodiment of a disposable hygiene protection pad 10 made in accordance with the present invention. As shown in FIG. 1, the pad 10 includes a first end 12, a second end 14, a positioning area 16 properly positioning the pad 10 to the wearer, and a plurality of thin layers of absorbent hygiene pads 15. Said plurality of pads 15 each having a first side 15A for facing towards the wearer's body, and a second side 15B for facing toward the undergarment of the wearer. Said pads 15 are individually formed of a lightweight, absorbent, breathable material that is fluid resistant, and is curved to conform to the shape of the wearer's body. It is understood that each of the said plurality of pads 15 are substantially identical in all respects.

The feminine hygiene pad 10 includes a longer design to accommodate the need that full-figured women have for a feminine hygiene napkin that covers more area than the current smaller designs, and may thus be more comfortably worn than prior art pads. As can be seen, the positioning area 16 is at a location in the approximate center of the protection pad 10. The first end 12 and the second end 14 having curved ends away from the positioning area 16. Said curved ends to better conform the pad 10 to the wearer's body. As shown in FIG. 1, the relative width of the positioning area 16 of the protection pad 10 is smaller than the width of the first and second ends 12, 14.

As best shown in FIG. 2, the layers of pads 15 include an interlocking means 20 preferably disposed along the outer perimeter of the first side 15A and second side 15B of the pads 15. The interlocking means 20 of the second side 1 5B of one of the pads 15 designed to receive the interlocking means 20 of the first side 1 5A of the next pad 15, so that each of said layers of pads 15 are releasably interlocked with the other. In the preferred embodiment, said interlocking means 20 extend around the outer perimeter of the first and second sides 15A and 15B of each of the layers of pads 15. It is understood that the interlocking means 20 for the first side 15A is a mirror image of the interlocking means 20 of the second side 15B of the layers of pads 15.

In the preferred embodiment as shown in FIG. 2, the interlocking means 20 consisting of a plurality of ribs 20A that interconnect as described above. In addition to the interlocking means 20, the layers of pads 15 are additionally removably affixed to one another at the ends 12,14, with any adhesive means suitable for bonding.

The pad 10 further includes a plurality of thin paper layers 30, wherein a single sheet of said paper layer 30 is situated and affixed to the second side 15B of each layer of pads 15, as shown in FIG. 1. In the preferred embodiment, said paper layers 30 shaped substantially identical to the shape of the pads 15, and sized in width and length slightly smaller than the pads 15 so that the paper layers 30 avoid obstructing the interlocking of the layers 15 as discussed above. It is understood the paper layers 30 further protect the layers of pads 15 below the first pad 15 being used, and serves as a barrier to prevent fluid from reaching the remaining pad layers 15. Although in the above description the shape of the paper layers 30 are described as substantially identical to the shape of the pads 15, it is understood that said paper layers 30 may be shaped to cover primarily the area of the positioning area 16, which area generally requires relatively greater absorbency.

In use, the protective pad 10 is positioned by the wearer with the top layer pad 15 in contact with the wearer's body. When the top layer pad 15 of the hygiene pad 10 needs changing, the wearer may simply peel away the top layer pad 15 with the paper layer 30 attached thereto, and have the remaining pad layers 15 of fresh sanitary protection underneath.

In the preferred embodiment, the layers of pads 15 are super absorbent hygiene protection pads currently available in the market. The paper layers 30 may be thin sheets of wax paper available in the market.

The protective pad 10 is formed of known materials. Preferably, the primary absorbency area in the area of the positioning area 16 is formed of a material of relatively great absorbency. The area surrounding the positioning area 16 may likewise be formed of a material of relatively great absorbency, or in the alternative, may be formed of a material that is not as absorbant.

Although the description above contains many specificities, these should not be construed as limiting the scope of the invention but as merely providing illustrations of some of the presently preferred embodiments of this invention.

Further, while the description illustrates the preferred embodiment as a feminine hygiene protection pad, a further use of the present invention may be for people who suffer from incontinence and use disposable undergarments, as well as infants using disposable diapers and panty pull-ups.

Thus the scope of the invention should be determined by the appended claims in the formal application and their legal equivalents, rather than by the examples given.

I claim:

1. A disposable hygiene protection pad comprising:
a plurality of thin layers of absorbent hygiene pads having a first side and a second side;
a plurality of paper layers;
an interlocking means disposed on the first side and the second side of the layers of pads;
an adhesive bonding means to removably affix the ends of the layers of absorbent pads;
wherein the second side of a first layer of absorbent pad is releasably interlocked with the first side of a second layer of absorbent pad;
wherein the paper layers are disposed between each of the layers of absorbent pads and affixed to the second side of each of the layers of absorbent pads.

2. The protection pad as recited in claim 1, wherein the interlocking means is disposed along the outer perimeter of the first side and the second side of the layers of pads.

3. The protection pad as recited in claim 1, wherein the layers of absorbent pads are formed of a lightweight, absorbent material that is fluid resistant.

4. The protection pad as recited in claim 1, wherein the paper layers are thin sheets of wax paper.

5. The protection pad as recited in claim 1, wherein the layers of pads having curved ends.

6. A hygiene pad comprising:
at least two thin layers of material having a positioning area formed of absorbent materials for contact with a wearer's body, said layers having a first side for facing towards the wearer's body and a second side for facing toward an undergarment of a wearer;
a paper layer in communication with the second side of each of said thin layers, said paper layer being relatively smaller in the dimension of length and width compared to the paper layers;
an interlocking means disposed on the first side and the second side of the thin layers; and
an adhesive bonding means to removably affix the ends of the thin layers.

7. The hygiene pad as recited in claim 6, wherein the interlocking means is disposed along the outer perimeter of the first side and the second side of the layers of pads.

8. The hygiene pad as recited in claim 6, wherein the first thin layer is releasably interlocked with the next thin layer.

9. The hygiene pad as recited in claim 6, wherein the thin layers are formed of a lightweight, absorbent material that is fluid resistant.

10. The hygiene pad as recited in claim 6, wherein the paper layer is a thin sheet of wax paper.

11. The hygiene pad as recited in claim 6, wherein the thin layers having curved ends.

12. A disposable protection pad comprising:
at least two thin layers of material formed of absorbent materials, said layers having a first side and a second side; and
a releasable interlocking means disposed on the first side and the second side of the thin layers, wherein the first thin layer is releasably interlocked with the next thin layer.

13. The protection pad as recited in claim 12, wherein the interlocking means is disposed along the outer perimeter of the first side and the second side of the layers of pads.

14. The protection pad as recited in claim 12, wherein the thin layers are formed of a lightweight, absorbent material that is fluid resistant.
